(19) United States
(12) Patent Application Publication
(10) Pub. No.: US 2011/0003354 A1
(43) Pub. Date: Jan. 6, 2011

(54) METHOD FOR CONVERTING HERBACEOUS PLANT FIBERS INTO FUEL ALCOHOL

(76) Inventor: Kaikwai Chan, Kowloon (CN)

Correspondence Address: Kaikwai CHAN, No. 424, Ta Chee Road, Taichung, 401, omitted

(21) Appl. No.: 12/884,309

(22) Filed: Sep. 17, 2010

Related U.S. Application Data
(63) Continuation-in-part of application No. 11/613,463, filed on Dec. 20, 2006.

(51) Int. Cl. C12P 7/02

(52) U.S. Cl. 435/155

(57) ABSTRACT

This disclosure teaches a method of converting herbaceous plant fibers into fuel alcohol comprising the following steps: The pre-treatment stage consists of grinding; using ultrasonic waves; adding the liquids mixed with alcohol, liquid ammonia, and water; adding NaOH; and then stirring and cooking. The second stage involves the recovery of organic liquids as well as high-pressure and high-temperature washing. Next, biological enzyme hydrolysis is conducted by adding endo-β-glucanase, exo-β-glucanase, and β-glucanase. Candida mycoderma, Rhizopus oryzae, ammonium sulfate, and phosphoric acid are added during the fermentation process. Finally, the alcohol is refined from distillate spirits, with further refinement in an alcohol tower.

### BACKGROUND OF THE INVENTION

This application is a Continuation-in-Part of application Ser. No 11/613,463, filed Dec. 20, 2006.

### FIELD OF THE INVENTION

This invention relates to methods of preparing alcohol, mainly referred to as a method of converting herbaceous plant fibers into fuel alcohol.

### DESCRIPTION OF THE PRIOR ART

At present, foods such as sorghum and corn serve as the main raw materials for producing fuel alcohol. The technology of producing fuel alcohol from corn consist of grinding the corn, mixing it with water in the proportion of from 1:30 to 1:40 corn to water by weight, heating the mixture to between 70 and 80° C., precooking for 20 to 40 minutes, and then boiling for no less than 90 minutes using a boiling vessel under 141 to 145° C. and pressure of from 3.2 to 3.5 kg/cm 2. However, many problems arise in using foods for the production of alcohol including high cost, low output rate, and high energy consumption.

Besides using foods, trials also have been made to produce alcohol using straws. The technology of such a method may consist of the following steps: grinding-syrup discharge-saccharification-sugar liquor-alcoholic fermentation-yeast separation-distillation-fuel alcohol. However, this method also has limitations such as high energy consumption and low output rate. Due to technical reasons, producing alcohol with straws is still at the experimental stage, which makes it unable to realize industrial production. Therefore, large numbers of straws and herbaceous plants are used as fuels, forages, and fertilizers, leading to a waste of resources.

The present invention has arisen to mitigate and/or obviate the afore-described disadvantages.

### SUMMARY OF THE INVENTION

The aim of this invention is to provide a method of converting herbaceous plant fibers into fuel alcohol. By adding cellulase and other materials, cellulose is hydrolyzed to reduce sugar, which then is fermented to produce fuel alcohol. This method improves the output and the speed of cellulose hydrolysis and overcomes the shortcomings of the present technology for fuel alcohol production.

The technical scheme of realizing this invention: this method comprises the following steps:

1. Pretreatment

a. Grind the raw materials of herbaceous plants into 100 to 180 mesh, and load them in a reaction tank.

b. Loosen and separate the structures between the walls of raw fibers by adding CO 2 with supersonic waves.

c. Add a liquid mixture of alcohol, liquid ammonia, and water to the raw material in the proportion of between 1:1.5 and 1:3 (the weight ratio of the raw material to the liquid). The alcohol accounts for 30-40%, and liquid ammonia accounts for 6-10%; the rest is water.

d. Add dilute alkali NaOH with 3-12% of the weight ratio of the raw material and stir.

e. Boil for 20 minutes to 2 hours at between 100 and 250° C. and the pressure of 2-6 Mpa.

f. Reduce the pressure to normal and reduce the temperature to between 30 and 40° C.

2. Recovery

a. Recover the organic liquids.

b. Wash them under high pressure and high temperature.

3. Biological Enzyme Hydrolysis

a. At 40 to 60° C. and pH 3.0 to 6.0, add the cellulase comprising endo-β-glucanase, exo-β-glucanase and β-glucanase. This accounts for 0.5 to 3% of the liquid weight

b. The enzymolysis process takes about 8 to 12 hours.

4. Fermentation

a. Under the temperature of 40-60° C. and pH 3.0-6.0, add liquid Candida mycoderma, Rhizopus oryzae, and dry yeast (compounded), which account for 3 to 8% of the liquid weight.

b. Ferment for 50-80 hours at the temperature of between 30 and 40° C.; and

c. Optionally, further add ammonium sulfate and phosphoric acid during this process to enhance fermentation.

5. Refinement

a. When the detected alcohol concentration reaches between 18 and 25 degrees, it will come into the preliminary process of refining alcohol from distillate spirits.

b. When the alcohol concentration reaches between 35 and 50 degrees, it will be sent to the alcohol tower for further refinement until the concentration reaches 95 degrees.

The technical scheme further includes:

The said cellulase further comprises glucosiduronate enzyme, acetyl enzyme, xylanase, β-xylanase, galactomannoglycan enzyme, and glucomannan enzyme.

The pressure/temperature relief process comprises transient decompression and transient cooling as well as real-time water-adding temperature relief.

The recovery of organic liquids comprises pretreatment using different alcohol, liquid ammonia and dilute alkali NaOH.

The beneficial results of this invention are chracterized by such features as extensive sources of raw material, simple process, low cost, low energy consumption, high output rate, environment protection and partial replacement of oil, this invention can also convert herbaceous plant fibers into other chemical products and biochemical products.

### DETAILED DESCRIPTION OF THE PREFERRED EMBODIMENTS

The present invention will be clearer from the following description when viewed together with the accompanying drawings, which show, for purpose of illustrations only, the preferred embodiment in accordance with the present invention.

### A. Pretreatment

a. Grind the raw materials of herbaceous plants into 100 to 180 mesh, and load them in a reaction tank.

This case technical feature is during carrying on the industrial production, when vegetable fiber raw material hydrolisis transfers the saccharification hydrolisis the effect, can enable in the saccharification hydrolisis fluid to have more glucose densities, as well as the less xylose density, is advantageous the efficiency which ferments in the enhancement, produces more ethanols. Simultaneously the equipment and the technology to create the output cost the influence, and the production technology's circulation again use influence output and the cost, and may recycle use again, and will not have the environmental pollution technical feature main point to take the patent research which will carry on to the overall technology's coordination.

1. Grind the raw materials of herbaceous plants into 100 to 180 mesh, and load them in a reaction tank,—The related attrition part of this application document is completely different from the levigation type processing raw material and general dry-like attrition processing raw material; The levigation type and the dry-like attrition as following:

The Potency of the Dry-Like Attrition
(1) The dry-like attrition energy consumption breakage great cost is very high, is the levigation-like five to six time of energy consumption quantities,
(2) The output by the dry-like attrition is very few,
(3) The dry-like milling equipment components lose the quick cost to be very high, the replacement loss components cost is very high,
(4) Dry-like grinds the complete set of equipment to be very expensive,
(5) The dry-like attrition dust is very big, the easy environmental pollution to need to reduce the dust,
(6) The dry-like attrition needs to increase pulls out vacuum cleaning, will otherwise have the environmental pollution cost to be very high, and will cause when the fermentation will have the fungus category pollution, will affect the production output, will make the cost to increase,
(7) The dry-like attrition can produce the very massive hot air, to the low temperature factory, has the cost to be very high,
   (1∘) the cost of The air conditioning increases,
   (2∘) the cost of Ferments Temperature controls increases
(8) When dry-like attrition production is limited to vegetable fiber chain's tearing, to reduces the following pretreatment the processing hydrolisis and the saccharification time has not helped,

The Potency of Water Type Attrition
(1) The water type attrition energy consumption quantity is low, compared to dry-like energy consumption quantity low five to six times,
(2) The water type attrition output is very big, the production output may increase along with the output demand, the supplemental equipment, the cost is low,
(3) The water type milling equipment components lose the few costs to be low, the replacement loss components few costs are low,
(4) The water type grinds the complete set of equipment to be cheap, dry-like grinds the complete set of equipment cost to be low, the expansion development is quick,
(5) Water type attrition dust if needs to reduce the dust very greatly,
(6) When water type attrition production does not need to increase pulls out vacuum cleaning, belongs to dustlessness type production, will not have the environmental pollution to create this increases, to fermentation when will not have the fungus category pollution, will not affect the production output, will make the cost to increase,
(7) The water type attrition cannot produce the massive hot air, will not produce to the low temperature factory controls the influence warm, will make the production cost to increase, (1∘) the cost of the air conditioning will not increase, (2∘) the cost of Ferments Temperature controls will not increase,
(8) When water type attrition production the biggest merit is carries on tearing to the vegetable fiber chain, reduces the following pretreatment the processing hydrolisis and the saccharification time, when the production already produced the heat generation high temperature cellulose to decompose, appearance “preliminary” cellulose hydrolisis,

b. Loosen and separate the structures between the walls of raw fibers by adding CO₂ with supersonicwaves:

The influence to cellulose by the pretreatment with carbon dioxide & ultrasonic wave jointly

Joins the carbon dioxide belongs to when the fermentation produces the product, will not have the situation which the cost will increase, after vegetable fiber attrition, will need to further carry on tearing again to the textile fiber chain, will carry on the ultrasonic wave using the recycling carbon dioxide coordination application ultrasonic wave to add the carbon dioxide to the vegetable fiber “second step” the pretreatment hydrolisis, the carbon dioxide coordination supersonic wave energy force cellulose decomposition will release the more monosaccharide members and the return to original state sugar density in the research is high, maximizing. The cellulose material uses the carbon dioxide coordination ultrasonic wave to increase separately to the cellulose demolition responded, thus raised the disintegration rate and the cellulose hydrolisis degree. In this pretreatment process, if micrite cellulose, when the use carbon dioxide coordination ultrasonic wave carries on the compression response to occur. When an explosive carbon dioxide relief pressure, and increased the cellulose substrate contacted area to the cellulose disarrangement of the structure, will affect to the hydrolisis effect is very big. The result indicated that the carbon dioxide coordination ultrasonic wave to the cellulose pretreatment is quite effective. In the pressure will increase will be advantageous in the crystal structure the carbon dioxide member seepage speed, after the detonation will occur, will thus produce more glucose is from the cellulose material separation. This detonation pretreatment enhanced the cellulose raw material hydrolisis rate as well as reaches as high as 50% glucose output increase. Also indicated from the synchronization diastatic fermentation test result that the fermentation cellulose may make the ethanol productivity for the carbon source material to increase. As one new substitution method, the plant lignocellulose demolition which the carbon dioxide coordination ultrasonic wave produces explodes, pretreatment law and so on airing, high temperature high pressure, low temperature low pressure compared to the ammonia to be able to reduce more expenditures and the time.

In the pretreatment technology of plant lignocellulose, has (1) the acid (H2SO4) pretreatment law, (2) the alkali (NaOH) pretreatment law, (3) the acid-ultrasonic wave union pretreatment law, (4) the alkali-ultrasonic wave union pretreatment law four pretreatment methods, in the research discovered that the acid and alkali treatment and the acid-alkali-ultrasonic wave joint treatment law density is 2.0%, the time is 60 min, but adopts jointly using the carbon dioxide ultrasonic wave in the processing law technology the power is 125W, the time is 20 min. Then the pretreatment processing vegetable fiber main chemical composition which adopted jointly after these four pretreatment method and the carbon dioxide ultrasonic wave has carried on the analysis separately, discovered adopted the pretreatment vegetable fiber jointly after the carbon dioxide ultrasonic wave, after its processing chemical constituent and the pure acid and alkali and the acid-alkali-ultrasonic wave joint treatment law pretreatment's vegetable fiber processing chemical composition compared, discovered adopted the pretreatment vegetable fiber after the carbon dioxide ultrasonic wave to be able jointly to have more cellulose and the hemicellulose content as well as the lignin content are less. Simultaneously the carbon dioxide coordination supersonic wave energy force cellulose decomposition releases the more monosaccharide members and the return to original state sugar density is high, then to passed through the pretreatment later vegetable fiber which four method pretreatments and the carbon dioxide ultrasonic wave adopted jointly to carry on the saccharification hydrolisis condition orthogonal to optimize, has obtained the final best technological conditions is: pH value 4.8, temperature 45° C., enzyme density 20 mgg−1. Then carried on to under the best technological conditions' saccharification hydrolytic process observed and analyzes, discovered that after the acid-ultrasonic wave pretreatment as well as the alkali-ultrasonic wave pretreatment's vegetable fiber can later achieve the stable return to original state sugar density under the best technological conditions after 108 h, and the maximizing, respectively was 26.4 gL−1 and 33.0 gL−1, but after the pure acid pretreatment as well as the caustic pretreating vegetable fiber must be able to cause the return to original state sugar density after 120 h to stabilize and to reach the maximum value, respectively was 26.2 gL−1 and 32.7 gL−1. But the carbon dioxide ultrasonic wave adopts jointly the pretreatment later vegetable fiber can later namely achieve the stable return to original state sugar density under the best technological conditions after 87 h, and maximizing 56.2 gL−1, has carried on the analysis finally after several kind of pretreatment method processing vegetable fiber saccharification hydrolisis fluid's main chemical composition, generally speaking, the use carbon dioxide ultrasonic wave pretreatment's vegetable fiber's saccharification hydrolisis effect must surpass the traditional pure acid, the alkali and the acid-alkali-ultrasonic wave joint treatment method pretreatment law, the use carbon dioxide ultrasonic wave pretreatment vegetable fiber saccharification hydrolisis effect can enable in the saccharification hydrolisis fluid to have more glucose densities as well as the less xylose density, is advantageous the efficiency which ferments in the enhancement, produces more ethanols. (after the pretreatment carbon dioxide and the ultrasonic wave may use again repeatedly, save the cost.):

c. Add a liquid mixture of alcohol, liquid ammonia, and water to the raw material in the proportion of between 1:1.5 and 1:3 (the weight ratio of the raw material to the liquid). The alcohol accounts for 30-40%, and liquid ammonia accounts for 6-10%; the rest is water. Add dilute alkali NaOH with 3-12% of the weight ratio of the raw material and stir.

d. Boil for 20 minutes to 2 hours at between 100 and 250° C. and the pressure of 2-6 Mpa.

e. Reduce the pressure to normal and reduce the temperature to between 30 and 40° C.

B. Recover the Organic Liquids: Wash them Under High Pressure and High Temperature;

After Completing the Carbon Dioxide Coordination Ultrasonic Wave Pretreatment, Uses Chemistry to Pretreat Again

Put the 95% alcohols, the liquid ammonia, and the moisture content by 40%, 6%, 54% do join into the reactor; and will dilute the alkalinity sodium hydroxide dilutes and carries on the agitation by 3-12% percents by weight.

“third step” the hydrolisis use sodium hydroxide (NaOH) dissolves in the ethanol and in the water altogether resolver mixture.

The findings indicated that after NaOH pretreatment, the vegetable fiber obtains the Run bulge obviously, the cellulose crystalline indice reduces, the cellulose crystallizing field receives the destruction, passes through cellulase processing after again, the crystalline indice has the enhancement: The NaOH pretreatment is one effective vegetable fiber pretreatment method, after NaOH pretreatment vegetable fiber change in enzymolysis.

In the research only uses 2% NaOH in 85° C. to process 75 min to the vegetable fiber, lignin removing rate achieves 47.9%, after the processing textile fiber residual is degraded easy by the enzyme, the enzymolysis rate achieves 33.2%; After the liquid-solid separation's lye may the reuse 3-4 times, thus reduces the pretreatment cost; The sodium hydroxide pretreatment condition is temperate, the energy consumption is low, and might remove in raw material fermentation inhibitors effectively and so on ethanoic acid, was advantageous in the vegetable fiber hydrolisis fluid ethanol fermentation.

The alkali treatment can enhance the cellulase effectively the function substrate—cellulose proportion, thus raises the enzymolysis sugar yield. but in this application document, transforms the herb the fuel alcohol the method use ethanol, the liquid ammonia and the water removes by organic mellow method the lignin the effect is quickest. In the organic acid resolver escapes in the lignin process, the lignin macro-molecule after joining alkalinity Qingyanghuana catalyzes the hydrolisis, causes the cellulose many linear high polymer glucose glucoside key's connection to be able to have the break, causes on the cellulose member chain to form the carbonyl, had the carbonyl cellulose not to be unstable, promoted the glycosidic linkage in alkazid solution break, reduced the polymerization degree. Uses the hydroxidized sodium ion liquid union use ethanol, the liquid ammonia and the water and so on by organic makes the precipitating agent mellowly, can transfer the cellulose reducibility end terminal grouping the carboxyl group, can also transform the mellow hydroxyl the carbonyl, then has the sugar glucoside key's break in the hot alkaline solution.

The cellulose high polymer polymerization degree drops, the solubility enhances, finally obtains the low molecular compound, this process is called the cellulose the degradation reaction.

Cellulose 1-4—The glucoside key is one kind of acetal key, the para acid is sensitive, under the suitable hydrogen ion concentration, the temperature and the time effect, the glucoside key break polymerization degree drops, this kind of response is called the cellulose the acidic hydrolysis, after the partial hydrolisis's cellulose product is called the hydrocellulose (hydrocellulose), time the complete hydrolisis's product produces the glucose.

Causes the lignin the soluble enhancement, uses the ethanol to take the precipitating agent, may promote the enzymolysis saccharification, the saccharification speed to compare the raw sewage cellulose not to enhance 80%.

The sodium hydroxide is the complete ion, includes the sodium ion and the hydroxide ion. The hydrogen ions way realizes the sodium hydroxide strong alkali and the acid response, dissolves in the sodium hydroxide this kind of ionic liquid the cellulose may through the make-up water, the ethanol and so on organic solvent separate out, because this is forms the hydrogen bond for this kind of resolver change and the hydroxidized sodium ion liquid, thus causes the cellulose instead to gather. May see when the findings, uses the ethanol makes the precipitating agent the obtained regenerated cellulose polymerization degree to be low, the saccharification speed is quick.

Meanwhile may see in the findings in 85° C. above the high temperature may destroy the cellulose intermolecular hydrogen bond, causes the dissolution; In 100° C. above insoluble cellulose; 1-butyl-3-methyl imidazole muriate ([BMIM]Cl) and 1-allyl-3-methyl imidazole muriate ([AMIM]Cl) the hydroxidized sodium ion liquid union use ethanol, the liquid ammonia and the water and so on by organic make the precipitating agent mellowly, including the strong hydrogen bond acceptor Cl-ion, causes the dissolution through them with the cellulose hydroxyl function. The carbaminate system is through with the cellulose in 100° C. above responded that transforms for the cellulose carbaminate, then dissolves again in the NaOH peroxide solution; The sodium hydroxide/water system, can only dissolve the crystallinity and the polymerization degree low cellulose; NaOH/liquid ammonia, peroxide solution they in precooling to −5—after 12° C., may dissolve the cellulose rapidly. Is mainly produces the small member through the low temperature and the greatly intermolecular new hydrogen bond network architecture, causes the cellulose intramolecular and the intermolecular hydrogen bond destruction dissolves, simultaneously the liquid ammonia the object impediment cellulose member causes the cellulose solution as Bao Hewu from the accumulation to be stable.

The findings indicated that uses the hydroxidized sodium ion liquid union use ethanol, the liquid ammonia and the water and so on by organic makes the precipitating agent mellowly, in 85° C. processes 35 min to the vegetable fiber, lignin removing rate achieves 87.2%, after the processing textile fiber residual is degraded easy by the enzyme, the enzymolysis rate achieves 93.7%; Because the sodium hydroxide this kind of ionic liquid union use ethanol, the liquid ammonia and the water and so on by organic make the precipitating agent mellowly, is the cellulose direct resolver, the fast precipitation may prevent dissolved the cellulose to restore to the original crystalline state. With the aqueous phase compared to, takes when the precipitating agent by the ethanol the cellulose separates out is more rapid. Moreover, the ethanol boiling point is low, may make the sodium hydroxide this kind of ionic liquid to be easier to recycle.

The findings indicated that in the cellulose enzyme hydrolysis vegetable fiber, has carried on the analysis to the cellulose saccharification advancement and the cellulase function mechanism. The result indicated that substrate enzymolysis in rate and pretreatment process lignin removing rate performance forward relevance, but the hemicellulose removing degree relations are not big; In the cellulose hydrolytic process at the same time the substrate hemicellulose becomes by the enzyme preparation in xylanase hydrolisis monosaccharides and so on xylose; In view of the textile fiber substrate specific volume major characteristic, uses makes up the material enzymolysis craft to enhance the substrate concentration in turn, thus enhanced in the enzymolysis fluid return to original state sugar density effectively, and has maintained the very high enzymolysis rate, caused the following fermentation the ethanol density to lay a better foundation.

C. Biological enzyme hydrolysis: At 40 to 60° C. and pH 3.0 to 6.0, add the cellulase comprising endo-β-glucanase, exo-β-glucanase and β-glucanase. This accounts for 0.5 to 3%o of the liquid weight The enzymolysis process takes about 8 to 12 hours.

The Biological Enzyme Raise and the Hydrolisis Transform Alcohol

First, we choose the fine mold mushroom spawn—mold mushroom spawn to have reproduce, the culture medium ingredient economy, to produce the enzyme stable property, the enzyme quickly thickly easy to separate characteristics and so on purification.

The next culture medium's configuration has the importance, the culture medium is provides the microorganism to grow the nutrients which, the reproduction and metabolism as well as the synzyme need. Therefore, culture medium pH, conditions and so on nutrients' ingredient and proportion must be advantageous to the protein synthesis. How to adjust the mold mushroom spawn reproduction condition introduction to be as follows:

Carbon source The carbon source is the microorganism cell structure as well as the vital activity material base, is one of synzyme primary data. Often uses each kind of starch and the hydrolysate like dextrin, the glucose and so on takes the carbon source.

Nitrogen source The nitrogen source is composes the protein and the nucleic acid principal element, is microorganism essential important raw material. The organic nitrogenous mainly has agricultural by-products as well as the protein peptone and so on bean cake, peanut cake material and so on. The inorganic nitrogen mainly has the ammonium sulfate, the ammonium chloride, the ammonium nitrate, the urea and so on.

Inorganic salts The inorganic salts constitute the microorganism cell's important ingredient, plays is adjusting the microorganism vital activity the role, mainly has the phosphorus, the sulfur, the magnesium, the potassium, the calcium, the copper, the iron, the manganese and so on.

Growth factor The growth factor is refers to the adjustment microorganism metabolic activity the micro organic matter, like Vitamin, amino acid, purine alkali, pyrimidine base and so on. In the enzyme production, the right amount increase growth factor may obviously raise the output.

PH The suitable potential of hydrogen is the microorganism normal growth as well as produces the enzyme the essential condition. Generally speaking, the most bacteria, the ray fungi grow most suitable pH is the neutrality to the alkalescency, but mold, yeast by chance slight acidity.

By liquid zymotechnics liquid in-depth zymotechnics. The enzyme preparation separation is refers to the enzyme withdraws from organism, causes it to separate with the impurity achieves the purity which the use goal adapts; Prevents the enzyme denatured deactivation. In obtains in enzyme extracting, the existence includes: Enzyme, mixed protein basis enzyme and mixed protein nature difference, purification method: 1, the centrifugal separation 2, filtrations and the membrane separation 3, precipitations separate 4, chromatographic analyses to separate.

β-glucosan Enzyme and β-grape glycosidase Assistance Hydrolisis Plant Cellose Rate Enhancement

In cellulose enzyme hydrolysis vegetable fiber process, biological enzyme hydrolysis processing: The temperature for 40-60° C., PH is 3.0-6.0, the increase β-glucosan enzyme and β-grape glycosidase may cause the fermentable monosaccharide rate enhancement, is one effective method of reducing the enzyme using amount. The reuse β-glucosan enzyme and β-grape glycosidase and optimized β-glucosan enzyme and β-grape glycosidase high production fungus raise condition and to produces the enzyme process to carry on the pH value and to make up the material regulation; The separation has purified outside two kind of butchers β-glucosan enzyme and β-grape glycosidase enzyme and one kind of butcher β-glucosan enzyme and β-grape glycosidase, matches the synthase the characteristic; Has established by the ultrafiltration, the acetone precipitation method, the fossilization enzyme law recycling use β-glucosan enzyme and β-grape glycosidase; And Beta-grape glycosidase carries on screening to β-glucosan enzyme, obtains β-glucosan enzyme and β-grape glycosidase high production strain, the maximum value achieves each strain 5.7 U/ml. In the secretion β-glucosan enzyme and β-grape glycosidase's process, the strain can produce the superelevation vigor interior contact β-glucosan enzyme and β-grape glycosidase and circumscribes β-glucosan enzyme and β-grape glycosidase, the filter paper enzyme achieves 0.75 IU/ml exactly. And, in the thick enzyme fluid β-grape glycosidase and the interior contact glucosan enzyme have strongly bear the ethanol characteristic. Produces when the enzyme controls the initial pH value is 5.0. Will produce the enzyme time the pH value control to be advantageous about 4.0 to β-glucosan enzyme and β-grape glycosidase secretion, when 100 h the enzyme vigor achieves each strain 6.15 U/ml. In produces in the enzyme process to pass through five supplement brans, the enzyme vigor enhanced nearly 4 times. Makes up in the material fermentation process, the suitable bran initial density is 2.8%, when makes up the material total quantity is certain, uses decreasing progressively to make up the material way effect to be best, its enzyme vigor achieves 7.35 U/ml. Makes up the material raise is in turn one kind very good raises the enzyme output the method. Uses steps and so on salting out, sparse water chromatographic analysis, anionic exchange chromatographic analysis, gel filtration, outside two kind of butchers who the purification obtains Beta-grape glycosidase, its molecular weight respectively is 122.3 KD and 80.7 KD. The macromolecular weight enzyme has bears the high sugar characteristic, take causes two kind of enzymes to the nitrophenol-β-D-glucose glucoside as the substrate not to need to rely on the metallic ion to maintain its activeness, can by certain density ethanol, the methanol, the normal butyl alcohol, the ethyl acetate activation. After the purification, in the butcher Beta-grape glycosidase, its molecular weight size is 135.2 KD. The methanol, the ethanol, the normal butyl alcohol, the acetone and the ethyl acetate and so on organic solvent has the remarkable activation function to this enzyme. Precipitates 2 h with −20° C. the acetone, the enzyme returns-ratio is 95.7%. When successive sedimentation cellobiose enzymolysis fluid, the 15th time enzyme returns-ratio still could about 46%, be possible to save needs the enzyme quantity 82.35%. When take the input speed as 1.5 ml/min, 1.0 the ml/min continual enzymolysis, the enzymolysis rate respectively achieves 96.65% and 99%. With uses the cellulase to compare alone, in β-glucosan enzyme and β-grape glycosidase total activity and the filter paper enzyme lives the ratio is 0.5 (FPA=2.0 IU/ml) under the condition, the fossilization β-glucosan enzyme and β-grape glycosidase and the cellulase synergism degeneration filter paper cellulose and micrite cellulose 60 h, their hydrolisis rate increased 28.4% separately and 33.1%.

β-glucosan Enzyme and β-grape glycosidase and xylanase Strengthening Cellulase—the Hydrolitic Reaction, the Sugar Rate Enhances Effect More Obvious

Uses the cellulose enzyme hydrolysis vegetable fiber in the research, discovered that spikes the xylanase to be able to strengthen the cellulase hydrolisis vegetable fiber the situation. In the research the cellulose enzyme hydrolysis responded that the sugar rate and the enzyme amount used, the sugar rate and reaction time's relations and join the xylanase strengthening cellulase hydrolisis, in the response the sugar rate and the xylanase recruitment, the sugar under rate and the reaction time reciprocity. Discovered that the cellulose enzyme hydrolysis response increase ration the xylanase, β-glucosan enzyme and β-grape glycosidase and under the cellulase synergism degeneration, may be clear about the affirmation hydrolisis plant cellose rate relative enhancement. Will surpass filters the technology to apply in the enzymolysis process, uses half batch operation. Inspected in half batch operation, the cellulase vigor respectively is under 50, 100 FPIU/g conditions, the sugar rate and reaction time's relations, as well as compare the sugar rate change situation with the batch operation. Uses half batch operation the enzymolysis-ultra to filter the coupled system to produce the sugar regarding the enhancement rate to have the effect. Uses half batch operation, β-glucosan enzyme and β-grape glycosidase and the xylanase strengthening cellulase hydrolitic reaction, the sugar rate enhances the effect to be more obvious. In the cellulase vigor is 50 FPIU/g, the increase β-glucosan enzyme and β-grape glycosidase and the xylanase sum total quantity, when respectively is 25, 50, 80 IU/g, the sugar rate enhances 33.38 separately, 41.80, 23.27%. The cellulase vigor is when 100 FPIU/g enhances 41.88 separately, 52.86, 39.96%. In half batch operation, the xylanase strengthening cellulase hydrolitic reaction, the unit mass zymoprotein produces the sugar quantity distinct enhancement. When the cellulase vigor is 50 FPIU/g, the increase β-glucosan enzyme and β-grape glycosidase and the xylanase quantity respectively is 25, 50, 80 IU/g, the unit mass zymoprotein produces the glucose quantity to enhance 12.93 separately, 15.16, 21.83%; The unit mass zymoprotein produces the cellobiose slightly to have the enhancement. When the cellulase vigor is 100 FPIU/g, the unit mass zymoprotein produces the glucose quantity to enhance 18.36 separately, 22.93, 18.08%; The unit mass zymoprotein produces the cellobiose quantity to enhance 17.66 separately, 18.38, 19.42%.

Research hydrolyze function. In the cellulase component circumscribes β-glucosan enzyme (the C1 enzyme), the interior contact β-glucosan enzyme (the CMC enzyme) and the interior contact β-grape glycosidase has the different adsorption nature on the identical cellulose substrate, in substrate granularity factors and so on size, pretreatment condition, pH value and temperature has the different influence to the cellulase adsorption. Under specific enzymolysis condition (substrate quality score 10%, pH value 4.8,50 ° C.), C1 enzyme, CMC enzyme component main adsorption on vegetable fiber textile fiber substrate, but interior contact β-grape glycosidase component majority of dissociations in liquid phase. Using the cellulase adsorption characteristic, has realized the cellulase recycling multiplying in the vegetable fiber enzymolysis craft. When the vegetable fiber textile fiber substrate quality score is 10%, cellulase initial amount used for each gram substrate 15 FPIU, after enzymolysis 48 h, filters out the hydrolisis fluid, the retention cellulose residual and joins the fresh substrate, simultaneously supplements the interior contact β-grape glycosidase (each gram substrate 4 IU) and the few cellulases (each gram substrate 7.5 FPIU), continues enzymolysis 48 h, is so redundant carries on. Indicated in the tandem duplication 5 batch of test results: This enzymolysis craft is easy to do, the cellulase amount used may save 50%.

In enzyme hydrolysis period, the cellulose is degraded into by the cellulase can by the yeast or the bacterial fermentation ethanol return to original state sugar.

With the cellulose enzyme hydrolysis herb textile fiber's in cellulose production ethanol, the cellulase is the function relatively slow enzyme, materially is because they use the substrate are complex, is insoluble, with half crystal structure. The cellulase also needs to have the quite high vigor as well as the correlation in β-glucosan enzyme, outside β-glucosan enzyme and β-grape glycosidase's synergism can transform completely the cellulose the glucose.

Biological enzyme hydrolysis processing: The temperature for 40-60° C., PH is 3.0-6.0, joins the cellulase including in β-glucosan enzyme, outside β-glucosan enzyme, in β-grape glycosidase, outside β-grape glycosidase, joins the quantity to account for the liquid weight 0.5-3%o, the enzymolysis process is 8-12 hours; Fermentation: In the temperature for 35-50° C., PH is 3.0-6.0 simultaneously joins the liquid rayon yeast and the Meegan mildew yeast and the dry yeast, joins the quantity to account for the liquid weight 3-8%o, the fermentation, temperature for 30-40° C., the fermentation time is 50-80 hours, in the fermentative process joins the ammonium sulfate and the phosphoric acid;

D. Fermentation: Under the temperature of 40-60° C. and pH 3.0-6.0, add liquid Candida mycoderma, Rhizopus oryzae, and dry yeast (compounded), which account for 3 to 8%o of the liquid weight. Ferment for 50-80 hours at the temperature of between 30 and 40° C.; and, further add ammonium sulfate and phosphoric acid during this process to enhance fermentation.

Candida mycoderma, Rhizopus oryzae, and Dry Yeast can Simultaneously Pentose Sugar and Six Carbon Sugars in the Hemicellulose Ferment to Ethanol

When the yeast cell density is higher than 8 g/L and the xylose content is higher than the total sugar 20%, the easy metabolism's hexose the inhibitory action which produces to the xylose to be possible to eliminate. In 30° C.˜40° C. and under the limit ventilation's condition, 90% above monosaccharides obtained the use, the ethanol rate are the 0.63˜0.78 g/g consumption sugars. The anaerobic fermentation causes the xylose to use and the xylitol accumulation not completely, but the ventilation excessive causes the yeast cell to multiply and to reduce the ethanol rate. This mold mushroom spawn to the cellulose, the hemicellulose hydrolisis fluid (textile fiber hydrolisis fluid) the fermentation inhibitor has high endures patiently ability. The fermenting property research, discovers Candida mycoderma, Rhizopus oryzae, and dry yeast, no matter bears the ethanol heat-resisting performance aspect in the fermenting power to be superior and other mold mushroom spawns. Embarks the strain take Candida mycoderma, Rhizopus oryzae as the mutagenesis, through EMS, LiCI and the ultraviolet ray three factor compound mutageneses, as well as 60Co-γ the exposure induces mutation or chromosomal change repeatedly, unifies the TTC plate, the Du acorn tube fermentation and the liquid state rocker ferments third-level screening, obtains two superiority mutagenesis strain finally, the two ethanol production rate achieves 0.383±0.015 g/g and 0.377±0.017 g/g, approximately for theoretical value 77.16% and 78.33%. The confirmation experimental result indicated that the fermentation vegetable fiber saccharification fluid produced the ethanol ability original mold mushroom spawn Candida mycoderma, Rhizopus oryzae enhanced approximately 2 times, after simultaneously cellulose enzymolysis saccharification, carried on when the fermentation transformation ethanol to the enzymolysis saccharification fluid its ethanol rate may reach above equally 87-93% by Candida mycoderma, Rhizopus oryzae, and dry yeast.

E. Refinement: When the detected alcohol concentration reaches between 18 and 25 degrees, it will come into the preliminary process of refining alcohol from distillate spirits. When the alcohol concentration reaches between 35 and 50 degrees, it will be sent to the alcohol tower for further refinement until the concentration reaches 95 degrees.

Absolute Ethanol Purification

The ethanol structural formula is CH3CH2OH, is the alcohol type one kind, is the liquor essential component, therefore is called the ethanol, popular name ethanol. The chemical formula may also write is C2HSO11 or EtOH, Et represents the ethyl. The ethanol is the commonly used fuel, the resolver and the disinfectant, also uses in the chemical industry.

The ethanol physical property mainly concerns with its low-carbon linear chain mellow nature. In the molecular hydroxyl may form the hydrogen bond, therefore the ethanol viscosity is very big, is also inferior to the close relative molecular mass the organic compound polarity to be big. Under the room temperature, the ethanol is colorless flammable, and has the special fragrance volatile liquid.

λ=589.3 nm and 18.35° C., the ethanol refractive index is 1.36242, compared to Shui Shaogao.

The ethanol (ethanol) under standard state's density is 0.79 absolute ethanol (dehydrated alcohol) under the standard state density is 0.7893.

Alcohol-water blend 20° C. time dense:

The ethanol solution density (g/cm3) (20° C.)−every (g/cm3) includes the ethanol weight (%) the g-density (volume to compare %)=(1 degree=1% volume)

0.791-99.5-99.7

May calculate: Every (g/cm3) includes the ethanol weight (%) g

The density (volume compares %)=(1 degree=1% volume)

For example: The density is 0.791 g/cm3

Every (g/cm3) includes the ethanol weight: 0.791×99.5%=0.787045 g

Density: 99.7%=99.7

Density (gram/centimeter 3) Ethanol density (%)

Alcohol Proof

When the Proof of an alcohol solution is used, one is dealing with a solution of mainly ethanol: CH3CH2OH and some water.

The value of the Proof is exactly twice the percentage of alcohol in the solution.

Pure alcohol is 200 Proof.

A solution that is 50% alcohol would be 100 Proof.

2. The said cellulase further comprises glucosiduronate enzyme, acetyl enzyme, xylanase, β-xylanase, gal actomannoglycan enzyme, and glucomannan enzyme.

3. The pressure/temperature relief process comprises transient decompression and transient cooling as well as real-time water-adding temperature relief.

4. The recovery of organic liquids comprises pretreatment using different alcohol, liquid ammonia and dilute alkali NaOH.

While we have shown and described various embodiments in accordance with the present invention, it is clear to those skilled in the art that further embodiments may be made without departing from the scope of the present invention.

What is claimed is:

1. A method of converting herbaceous plant fibers into fuel alcohol, comprising the following steps: (a.) grinding raw materials of herbaceous plants to the size between 100 and 180 Mesh, and loading them in a reaction tank; (b.) adding CO₂ gas and applying supersonic waves; (c.) adding a liquid mixture of alcohol: liquid ammonia: water in a ratio of 30-40%:6-10%:50-64% by weight to the raw material in a proportion of between 1:1.5 and 1:3 of the raw materials to the liquid mixture by weight; adding dilute alkali NaOH in a proportion of 3-12% of alkali to the raw materials by weight, and stir the mixture; (d.) boiling the mixture for 20 minutes to 2 hours under the temperature of 100-250° C. and the pressure of 2-6 MPa; (e.) reducing the pressure to normal atmospheric pressure and reducing the temperature to 30-40° C.; (f.) optionally, recovering the organic liquids; (g.) under the temperature of 40-60° C. and pH between 3.0 and 6.0, adding aqueous suspension of cellulase comprising endo-β-glucanase, exo-β-glucanase and 3-glucanase which account for 0.5-3% of the liquid weight, and allowing a reaction to take place for 8-12 hours; and (h.) under the temperature of 40-60° C. and pH between 3.0 and 6.0, adding aqueous suspension of Candida mycoderma, Rhizopus oryzae and dry yeast which account for 3-8% of the liquid weight; optionally further adding ammonium sulfate and phosphoric acid; keeping the temperature at 30-40° C. and allowing reaction to take place for between 50 and 80 hours.

2. The method of claim 1, wherein when the alcohol concentration reaches between 18 and 25 degrees, the reaction mixture will be subjected to a preliminary process of refining alcohol from distillate spirits.

3. The method of claim 1, wherein when the alcohol concentration reaches between 35 and 50 degrees, the reaction mixture is sent to an alcohol tower for further refinement until the alcohol concentration reaches 95 degrees.

4. The method of claim 1, wherein the cellulase in step (g.) further comprises β-Glucoside acid enzyme, transacetylase, xylanase, β-xylanase, galactomannan enzyme and glucomannan enzyme.

5. The method of claim 1, further comprising transient pressure relief and real-time water-adding temperature relief.

6. The method of claim 1, wherein step (f.) further comprises a pretreatment using an alcohol which is different from the fuel alcohol, liquid ammonia and dilute alkali NaOH.
